(19) United States
(12) Patent Application Publication
(10) Pub. No.: US 2011/0208202 A1
(43) Pub. Date: Aug. 25, 2011

(54) POSITIONING AID IN THE FORM OF A SELF-AFFIXING TEST JIP JOINT SOCKET

(75) Inventors: Lukas Zumsteg, Sulz (CH); Thomas Nmi Kilchenmann, Horgen (CH)

(73) Assignee: SMITH & NEPHEW ORTHOPAEDICS AG, Memphis, TN (US)

(21) Appl. No.: 13/057,024

(22) PCT Filed: Jul. 23, 2009
(86) PCT No.: PCT/EP09/59502

§ 371 Date: May 6, 2011

(30) Foreign Application Priority Data
Aug. 1, 2008 (DE) 102008036036.8
Sep. 17, 2008 (DE) 102008047627.7

(51) Int. Cl. A61B 17/90

(52) U.S. Cl. 606/91

(57) ABSTRACT

The invention relates to a self-fixating trial socket for releasable insertion into a corresponding cavity in a hip bone. The self-fixating trial socket comprises an outer surface, which is matched to the cavity in the hip bone, an inner surface, which is matched to an inlay, especially a trial inlay, and/or to a ball joint head, for receiving the latter in the hip joint socket, especially self-fixating trial socket, and at least one holding element for fixating the hip joint socket in the cavity, the at least one holding element comprising at least one spring element which is constructed in such a way that in the inserted state the spring element exerts, at least in some places, an outwardly directed spring force on the cavity in the hip bone for releasable fixation of the hip joint socket.

The invention relates to a positioning aid in the form of a self-fixating trial hip joint socket.

Hip joint sockets are constituents of conventional hip joint prostheses, which include a femoral stem, which is implanted into the femoral bone, and a ball joint head which is anchored on the femoral stem, the ball joint head being rotatably mounted in the hip joint socket. The hip joint socket is usually implanted in the pelvis, there being implanted a shell-like, metallic anchoring body in which a plastics body, in which the hip ball articulates, is inserted.

The hip joint socket—as described, for example, in DE 197 14 050 A1—is usually securely joined to the hip bone by means of one or more anchoring pins or one or more screws. Further forms of anchoring the socket in the bone are fixation by means of a screw thread on the shell (EP 0 601 224 B1) or fixation by means of a press-fit, achieved by specific over-dimensioning of the metallic shell body relative to the prepared seat in the bone.

The relative position of hip joint socket and hip joint stem determines the range of movement of the joint within which the joint can be moved without impingement between the shank neck and the socket edge. Whereas the hip joint stem is fixed in its position essentially by the morphological conditions of the femoral bone, the orientation of the socket in the pelvis is freely selectable. In order to avoid or at least reduce the risk of dislocation of the hip joint head or damage to the socket as a result of impingement between a shank neck and the socket edge, impingement and dislocation tests are carried out during implantation of the individual components of the hip prosthesis. The risk of impingement can also be counteracted by changing the length of the shank neck. In this respect, reference is made by way of example to DE 10 2006 045 358 A1, in which an adjustable shank neck is described. Once the metallic socket shell has been implanted, the risk of impingement and dislocation can be influenced only to a limited extent by the use of inlays having differently configured edges. For selecting and determining the position of the definitive inlay there are used re-usable trial inlays which are positioned in the implanted socket shell in an easily releasable way.

The use of an adjustable shank neck or the use of different inlays is accordingly able to correct unsatisfactory positioning or orientation of the hip joint socket only to some extent. Such a correction is relatively laborious, however, and is considered overall to be unsatisfactory. If the correction involves the use of different inlays, it is, in addition, regarded as disadvantageous that the choice of inlay is made on the basis of criteria that limit its scope for variation.

The present invention is based on the problem of providing a positioning aid for implantation of a socket implant of a hip joint prosthesis with which the risk of dislocation is effectively reduced, while the patient is subject to as little stress as possible during the operation. That problem is solved by a positioning aid in the form of a self-fixating trial hip joint socket (hereinafter referred to as a “trial socket”) which can be operated using a manipulation instrument, in accordance with patent claim 1. The problem is further solved by an arrangement comprising the self-fixating trial socket and a manipulation instrument in accordance with claim 9, by an arrangement comprising the self-fixating trial socket and a navigation system having a position-determining aid and/or orientation-determining aid in accordance with claim 10, and by an arrangement comprising a self-fixating trial socket and an inlay, especially a trial inlay, and a set consisting of a plurality of trial sockets in accordance with claim 12.

The invention is based on the concept of providing a self-fixating trial socket for releasable insertion into a corresponding cavity in a hip bone. The self-fixating trial socket comprises an outer surface, which is matched to the bone cavity, an inner surface, which is matched to an inlay, especially a trial inlay, and/or to a ball joint head, for receiving the latter in the self-fixating trial socket, and at least one holding element for fixating the self-fixating trial socket in the cavity, the at least one holding element comprising at least one spring element which is constructed in such a way that in the inserted state the spring element exerts, at least in some places, an outwardly directed spring force on the cavity in the hip bone for releasable fixation of the self-fixating trial socket.

The self-fixating trial socket can be provided with a trial inlay which articulates with the ball head of the hip stem. Because ball heads of different diameter are used, the trial inlay can be joined to a self-fixating trial socket so as to be exchangeable, for example by means of a screw thread in the pole of the self-fixating trial socket. Usually, ball heads of 22 mm, 28 mm, 32 mm or 36 mm diameter are available. Accordingly, it is possible to provide trial inlays of those diameters which can be inserted into different self-fixating trial sockets.

Depending upon the choice and mounting of the trial inlay in question, the self-fixating trial socket can be positioned in the prepared bone cavity by means of a manipulation instrument and fixated by relaxation of the spring element/spring elements, for example one or more leaf springs.

The spring element exerts a holding force on the self-fixating trial socket, so that the latter is fixated inside the cavity in the hip bone. The self-fixating trial socket can easily be positioned in the cavity in the hip bone and removed therefrom again by using the manipulation instrument to apply a force opposing the spring force, i.e. the holding force. As a result, the self-fixating trial socket can be repeatedly positioned and oriented very exactly. For example, once the self-fixating trial socket has been inserted into the cavity in the hip bone using the manipulation instrument, various tests can be carried out, for example impingement and dislocation tests or a test of the scope for movement of the femoral bone. On the basis of the results of those tests, if they are positive, the definitive position and orientation of the self-fixating trial socket can be ascertained or, if the results of the tests are unsatisfactory, the position and/or orientation can be altered accordingly. That procedure can be repeated any number of times until a satisfactory result has been obtained, the use of the spring element avoiding damage to the hip bone. In particular, there is no need for fixating elements that cause damage to the hip bone, such as pins or screws. Once the definitive position and orientation has been ascertained using the self-fixating trial socket, the socket implant is implanted in the position and orientation so determined.

The transferring of the position and orientation so determined to the implant can be reliably effected, for example, with the aid of a surgical navigation system. For that purpose, for example, a suitable position indicator is placed on the manipulation instrument, which can be attached to the self-fixating trial socket. A position indicator can then likewise be placed on a manipulation instrument for the socket implant. The position of the socket implant can thus be selected to be exactly analogous to the position of the self-fixating trial socket that has been found to be optimum. Alternatively, the position can be reproduced on the basis of markings applied to the bone cavity. Such markings indicate, for example, the course of the edge of the self-fixating trial socket in the position that has been found to be optimum. The edge of the definitive socket implant can then be selected so that it is brought into register with the marking.

The present self-fixating trial socket therefore helps to avoid the risk of dislocation or impingement in an effective way that causes little stress to the patient.

In a preferred embodiment, the spring element or at least one of the spring elements is in the form of a leaf spring which, preferably at least in some regions, forms the outer surface of the self-fixating trial socket. Such a spring element is especially simple to produce and occupies only very little space inside the self-fixating trial socket.

Preferably, the proximal end of the leaf spring/leaf springs is fixed to the self-fixating trial socket in the vicinity of the piercing point of an axis of symmetry thereof, and the position of the distal end located in an edge region of the self-fixating trial socket is variable by being subjected to pressure. In such an arrangement of the leaf spring, the latter is stressed during the insertion operation. As a result, two operations, namely the insertion of the self-fixating trial socket and the stressing of the leaf spring, can take place simultaneously.

Preferably, the holding element(s) has/have protrusions, especially sawtooth-like protrusions, on a surface portion of the outer surface of the self-fixating trial socket, which surface portion faces the hip bone. This improves the anchoring of the self-fixating trial socket in the cavity in the hip bone.

The protrusions are preferably arranged at the distal end of the leaf spring. Relatively high pressure acts in the region of the distal end, for which reason the protrusions are especially effective in that region.

In a specific development of the self-fixating trial socket, in the inserted state a portion thereof projects preferably by at least 0.5 cm, especially by 1.0 cm, beyond the wall of the cavity in the hip bone. Such a projecting portion offers an engagement surface so that the self-fixating trial socket can be removed from the cavity in the hip bone without great effort by means of a manipulation instrument.

Preferably, the holding elements are configured in such a way in the region of the projecting portion that they can be brought into engagement with a corresponding manipulation instrument for releasing the holding elements. This facilitates removal of the self-fixating trial socket.

In a preferred embodiment, the self-fixating trial socket has through-apertures such that a grid-like structure is formed. As a result, the surgeon can make a visual assessment of whether the self-fixating trial socket is in snug contact with the prepared bone cavity and sufficient bone is available for supporting the metal shell that is to be implanted later.

The above-mentioned problem is further solved by an arrangement comprising the self-fixating trial socket and a manipulation instrument, the manipulation instrument being constructed in such a way, especially like forceps, that it is possible for a person or device operating the manipulation instrument to apply to the holding elements a force that is directed into the interior of the self-fixating trial socket. By the use of the manipulation instrument, especially in the case where the manipulation instrument is of forceps-like construction, a relatively high force can be applied to the holding elements, allowing the use of relatively “hard” leaf springs.

In other words, the above-mentioned problem is further solved by an arrangement comprising the self-fixating trial socket and a navigation system having a position-determining aid and/or an orientation-determining aid, the position-determining aid and/or orientation-determining aid being fixable to the self-fixating trial socket for determining and storing the position and/or orientation thereof. By means of such an arrangement, the position and/or orientation of the self-fixating trial socket can be exactly determined. This is advantageous when the self-fixating trial socket fulfils solely a trial function, that is to say after a suitable position and orientation has been determined it is replaced by a socket implant better suited to long-term use. Long-term attachment of this last-mentioned socket implant to the hip bone can then also be effected by means of screws, anchoring pins, or the like.

Furthermore, the problem is also solved by an arrangement comprising the self-fixating trial socket and an inlay, especially a trial inlay, which is insertable or inserted into the self-fixating trial socket. Such an arrangement can also be used to test different trial inlays.

Finally, the problem is also solved by a set consisting of a plurality of self-fixating trial sockets, the individual self-fixating trial sockets differing in size, especially in respect of their ball radius. The provision of such a set makes it possible not only to effect exact setting of the position and orientation of the self-fixating trial socket and thus ultimately of the socket implant, but also to subject their geometric configuration to different tests. Such a set can be augmented by the provision of different inlays, especially trial inlays.

Further embodiments will be found in the subsidiary claims.

Hereinbelow the invention will be described in respect of further features and advantages on the basis of exemplary embodiments which are explained in detail with reference to the accompanying diagrammatic drawings, wherein

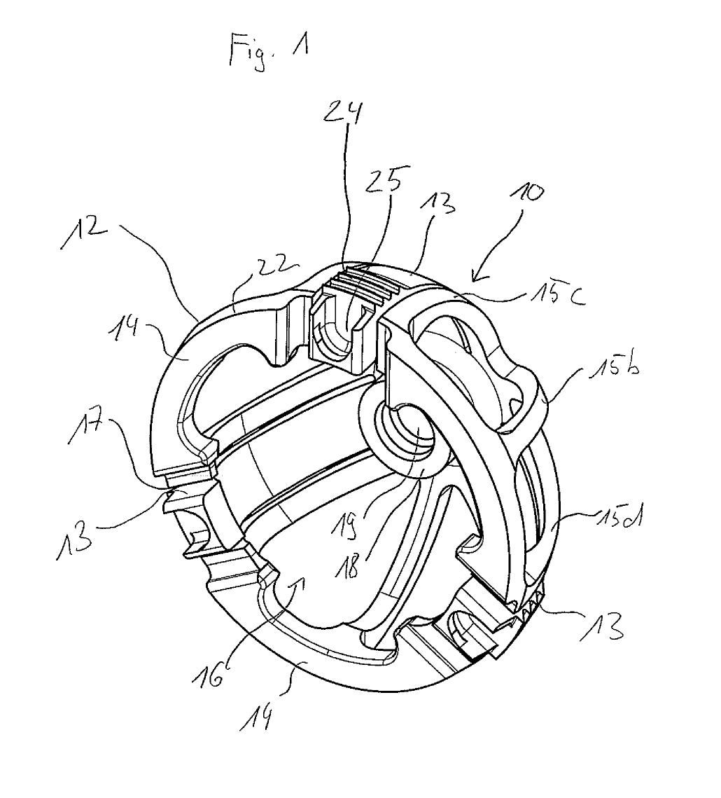

FIG. 1 is an oblique view of a self-fixating trial socket in accordance with a first embodiment;

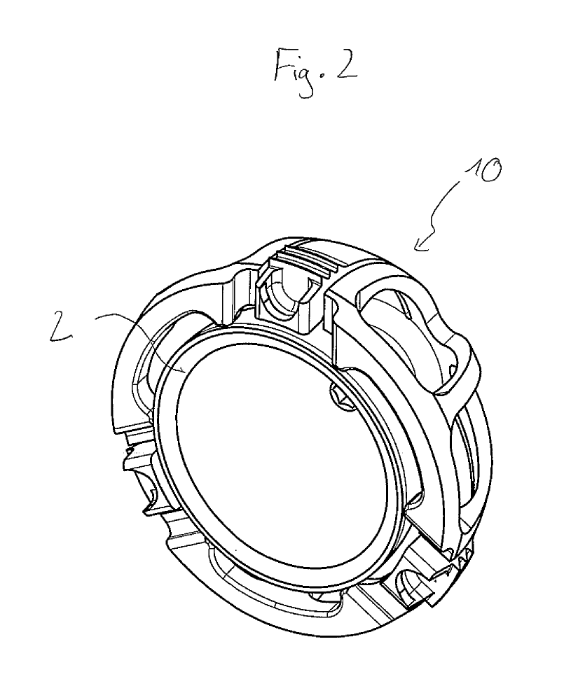

FIG. 2 is an oblique view of the self-fixating trial socket in accordance with the first embodiment with a trial inlay inserted;

FIG. 3 is a sectional view of the embodiment in accordance with FIGS. 1 and 2;

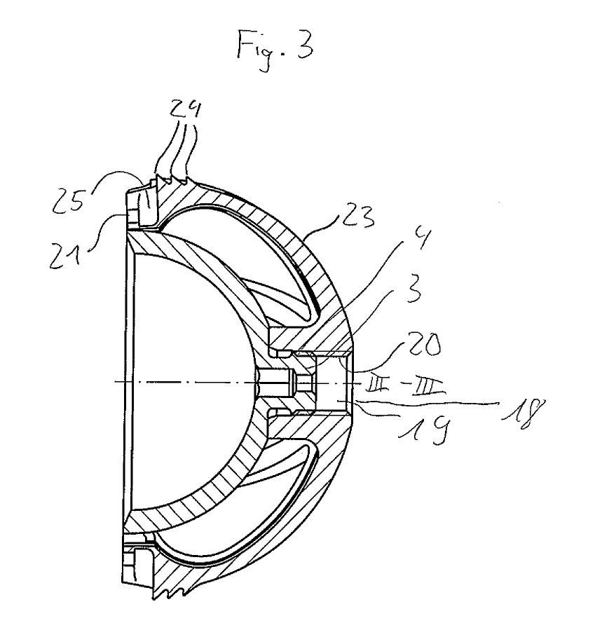

FIG. 4 is a section along line III-III from FIG. 3 with a holding element in a first position;

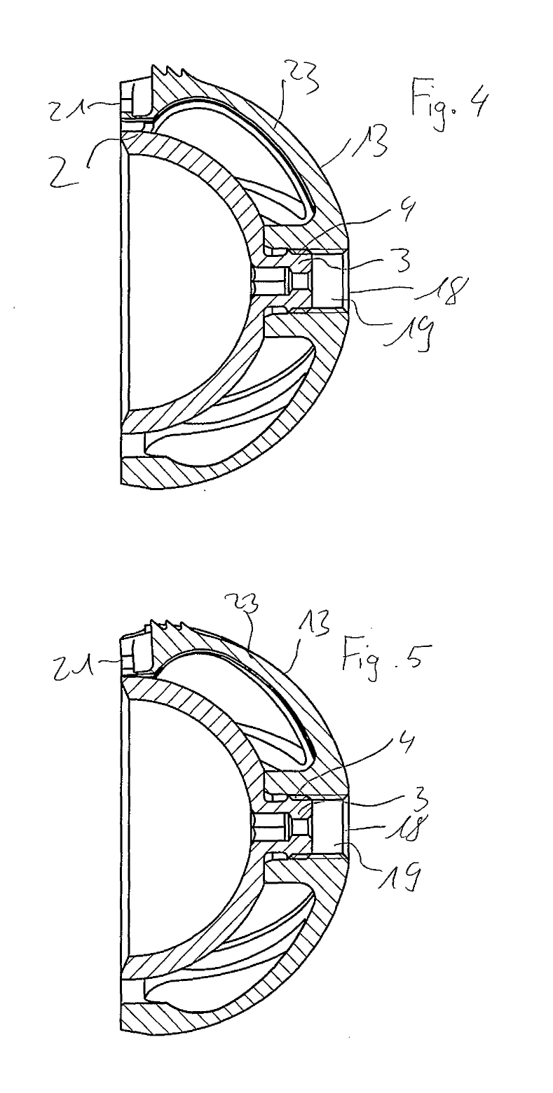

FIG. 5 is a section along line III-III from FIG. 3 with the holding element in a second position;

FIG. 6 is an oblique view of a trial inlay;

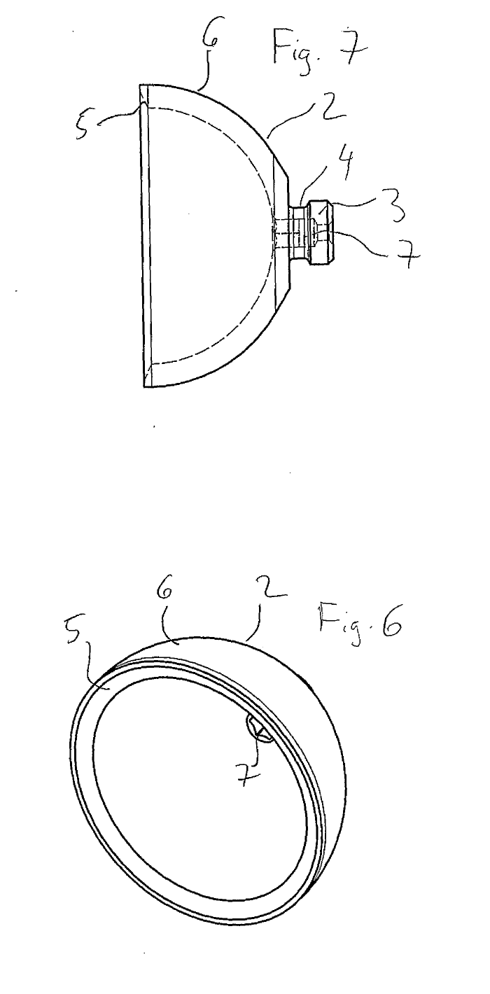

FIG. 7 is a sectional view of the trial inlay from FIG. 6;

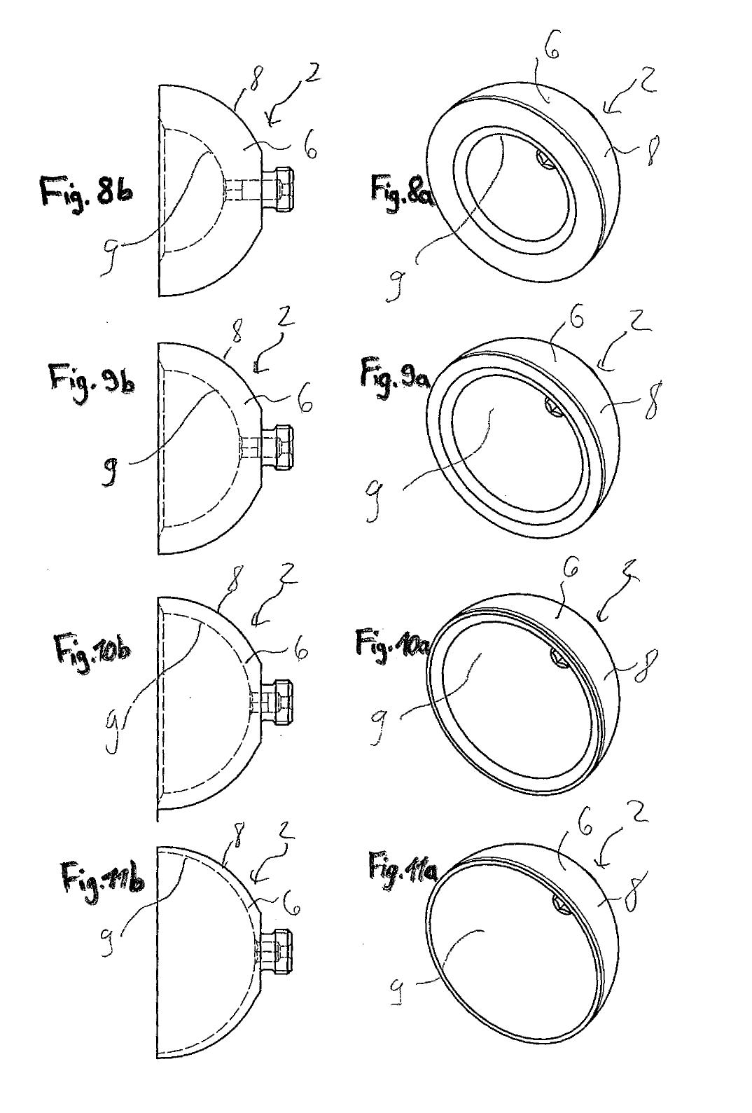

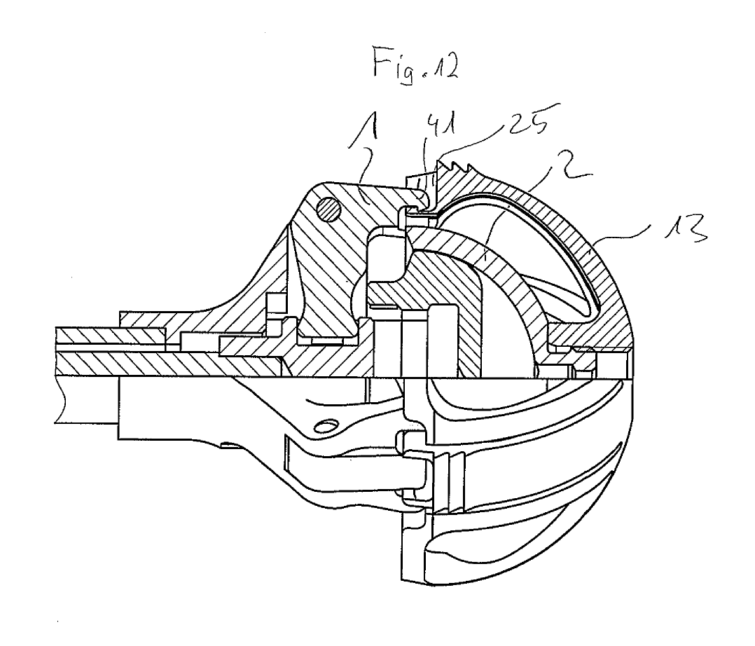

FIG. 8a-FIG. 11a are sectional views of different embodiments of the trial inlay;

FIG. 8b-FIG. 11b are sectional views of the trial inlays from FIG. 8a-10a;

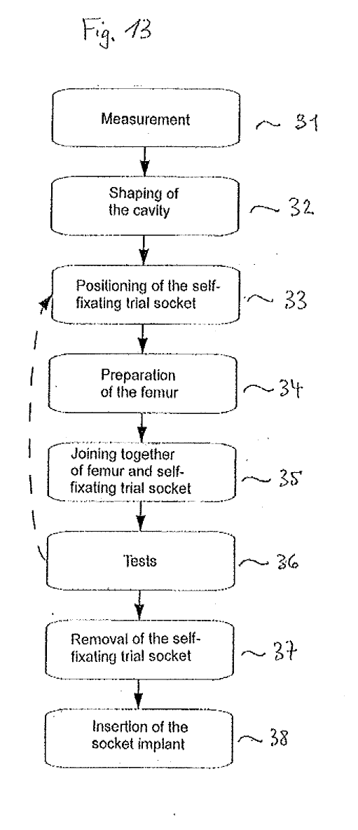

FIG. 12 shows a trial hip joint socket and a manipulation instrument (shown partly in section); and

FIG. 13 is a flow diagram.

In the following description, the same reference numerals are used for parts that are the same or act in the same way.

FIG. 1 is an oblique view of a self-fixating trial socket. The shape of the self-fixating trial socket corresponds approximately to a hollow hemisphere. When the self-fixating trial socket is in the inserted state, the outer surface 10 is, at least in some regions, in contact with a cavity in a hip bone (not shown in the Figures). The inner surface 11 serves for receiving an inlay or a ball joint head (inlay and ball joint head not shown in the Figures).

The self-fixating trial socket comprises a main body 12 and three holding elements 13. Alternatively, it is also possible for four or five or any number of holding elements (not shown in the Figures) to be provided.

The main body 12, which is preferably made of a metal or a metal alloy, especially of steel, preferably instrument steel, or a titanium alloy having suitable spring characteristics, forms an approximately circular pole portion 18 and three further portions 14. The pole portion 18 has a circular recess 19 which can be used, for example, for fixating an inlay (not shown in the Figures). The three portions 14 consist of four ribs 15a to 15d. The ribs 15a to 15c run approximately meridionally and are joined to one another by the rib 15d which runs equatorially in the region of a socket opening 16. The portions 14 of the main body 12 are spaced apart from one another, the holding elements 13 being located in intermediate spaces 17 which are bounded laterally by the portions 14.

In FIG. 2 the holding elements 13 are shown again separately in the form of a diagrammatic section taken radially. The proximal end 20 of the holding element 13 is securely joined to the pole portion 18 (see FIG. 1) of the main body 12. The distal end 21 is unattached and located in an edge region 22 of the self-fixating trial socket.

The holding element 13 (see also FIG. 3) comprises a leaf spring 23. Further constituents of the holding element 13 are the sawtooth-like protrusions 24 and the recess 25. The sawtooth-like protrusions 24 are provided at the distal end 21 of the holding element 13 and face in the direction of the cavity in the hip bone. The recess 25 is arranged at the distal end 21 of the holding element 13 in the region of the projecting portion (in the inserted state) of the self-fixating trial socket.

The sawtooth-like protrusions 24 increase the holding force between the self-fixating trial socket and the cavity in the hip bone into which the self-fixating trial socket is to be inserted. The recess 25 serves, for example, as a counter-part for a corresponding forceps-like manipulation instrument 1 (see FIG. 12), with the aid of which a force can be exerted on the three holding elements 13 in the (radially) inward direction. It is also possible for positioning and/or orientation aids as components of a navigation system to be fixed to the respective recesses 25 in the holding elements 13. The positioning and orientation aids can also be mounted in some other way, however, for example on the ribs 15d of the portions 14.

The self-fixating trial socket is constructed merely for test purposes during the operation. A socket implant is then used for long-term installation in the patient. Such a socket implant can be non-releasably joined to the hip bone, for example by cementing.

FIGS. 2 to 5 show the hip joint socket from FIG. 1 with the trial inlay 2 inserted. Using the trial inlay it is then possible to carry out a joint test with impingement monitoring. The trial socket is therefore preferably used in combination with the trial inlay.

FIGS. 4 and 5 show two different positions of the holding element 13 with the leaf spring 23. In FIG. 4, the distal end 21 of the holding element 13 is spaced apart from the trial inlay 2. The arrangement consisting of trial socket and trial inlay 2 is located in this position, for example, when the trial socket is installed in the cavity in the hip bone. In FIG. 5, in turn, the distal end 21 of the holding element 13 has been pressed radially inwards in comparison with FIG. 4. When all three holding elements 13 (of which only one can be seen in FIGS. 4 and 5) are in this position, the trial socket including the trial inlay can be removed from the cavity in the hip bone.

The trial inlay 2 (see FIGS. 3 to 5) is preferably fixed in the recess 19 of the pole portion 18 of the trial socket by way of a fixing element 3. In the present case, the fixing element 3 has an external thread 4 by means of which the trial inlay 2 can be screwed together with the trial socket. The external thread 4 can be made, for example, from steel.

In FIGS. 6 and 7, the trial inlay 2 is shown again on its own. The Figures also show, in addition to the fixing element 3 with the external thread 4 and a hollow space 7, the configuration of a main body 6 which has an edge 5. The main body 6 has the shape of a hollow hemisphere, like the main body 12 of the trial socket (not shown in FIGS. 6 and 7), and has no openings. The edge 5 is bevelled radially inwards.

FIGS. 8a to 11a and FIGS. 8b to 11b show four different embodiments of the trial inlay 2. The outer surface 8 of the main body 6 is the same in all four embodiments, so that the four trial inlays shown can be inserted into the same trial socket. In principle, it is, of course, also conceivable to provide trial inlays having different outer surfaces 8 in order that a variety of trial sockets can be subjected to the mentioned tests.

Preferably, inlays having a cup diameter of 22 mm, 28 mm, 32 mm or 36 mm are produced. The inlay having a cup diameter of 22 mm provides the smallest range of movement and the inlay having a cup diameter of 36 mm the greatest range of movement. It is especially advantageous for an entire set of different trial inlays to be available at the same time, for example during the operation. During the operation, a specially manufactured manipulation hip ball (not shown in the Figures) is preferably inserted into the trial inlays.

FIG. 12 shows the trial socket with an inserted trial inlay, the manipulation instrument 1 being in engagement with the trial socket. In more specific terms, gripping elements 41 are fitted into the recesses 25 of the holding elements 13. The manipulation instrument 1 is therefore constructed in such a way that the trial socket can be positioned and removed together with the inserted trial inlay. A further advantage, therefore, in addition to its stable position, which can be achieved by means of the screwed connection in the pole portion 18 (see FIGS. 3 to 5), is its easy exchangeability by means of the manipulation instrument 1. Using the manipulation instrument 1, a force can be applied to the trial socket in the radially inward direction.

FIG. 13 shows a flow diagram which illustrates the typical course of a hip operation with the aid of the self-fixating trial socket described in FIGS. 1 and 2. The operation described herein can be divided into eight sub-steps (represented by Blocks 31 to 38).

### Step 1 (Block 31):

In the first step, the hip region of the patient is measured, the position and orientation of the individual components in respect of the operating table being stored. Hereinbelow the term orientation/position always relates to the operating table as reference.

### Step 2 (Block 32):

In this (optional) step, the patient's hip bone is subjected to preliminary working such that the self-fixating trial socket is insertable.

### Step 3 (Block 33):

The self-fixating trial socket is then inserted into the cavity in the hip bone, taking into account the measured values from Step 1, the position and orientation of the self-fixating trial socket being determined and stored by means of a position and orientation determining device or a navigation system. If the position does not agree with the position calculated in Step 1, the self-fixating trial socket can be released and repositioned with the aid of a suitable manipulation instrument.

### Step 4 (Block 34):

A femoral stem is driven into the femur and then joined to the ball joint head by way of the shank neck.

### Step 5 (Block 35):

The ball joint head is then inserted into the self-fixating trial socket. A suitable inlay can be selected beforehand, perhaps from a range of inlays, and inserted into the self-fixating trial socket.

Step 6 (block 36):

Once the femur and the self-fixating trial socket have been joined together, various tests, such as, for example, a range of movement test, an impingement test and a luxation test, are carried out. If the results of these tests are satisfactory, Step 6 is followed by the seventh step. If the results are unsatisfactory, the surgeon returns to Step 3 and changes the position of the self-fixating trial socket.

### Step 7 (Block 37):

Using a suitable manipulation instrument, which can also be used for positioning the self-fixating trial socket, the trial socket is then removed. It will be understood that here it is especially important for the exact position and orientation of the self-fixating trial socket to be determined.

### Step 8 (Block 38):

In this step, a socket implant is implanted, while maintaining the previously determined ideal position of the self-fixating trial socket. The socket implant is, for example, cemented in place and/or screwed together with the hip bone.

In Step 3 (positioning of the self-fixating trial socket) it is also conceivable for the different self-fixating trial sockets, for example of different radius, to be inserted and tested in Step 6. In the same way, in Step 5 it is conceivable for different inlays, especially trial inlays, to be provided in order that they can be tested in Step 6.

Alternatively it is also possible to insert into the cavity in the hip bone an intermediate shell in which the self-fixating trial socket and/or the socket implant is/are positioned.

It is especially advantageous for the self-fixating trial socket to project by a certain amount relative to the cavity in the hip bone. This makes it easier to bring the manipulation instrument and/or a position-determining aid and/or an orientation-determining aid into engagement with the self-fixating trial socket.

Although the present invention is intended especially for use in the human body, it can also be used in animals, especially dogs, and constructed accordingly.

It should be pointed out here that all the parts described above, considered on their own or in any combination, especially of the details shown in drawings, are claimed as being important to the invention. Modifications thereof are familiar to the person skilled in the art:

### LIST OF REFERENCE NUMERALS

1 manipulation instrument
2 trial inlay
3 fixing element
4 external thread
5 edge (of the trial inlay)
6 main body (of the trial inlay)
7 hollow space (of the trial inlay)
8 outer surface (of the trial inlay)
9 inner surface (of the trial inlay)
10 outer surface
11 inner surface
12 main body
13 holding element
14 portions
15a-d rib
16 socket opening
17 intermediate spaces
18 pole portion
19 recess (in the pole portion)
20 proximal end
21 distal end
22 edge region
23 leaf spring
24 protrusion
25 recess (in the holding element)
30-38 block
41 gripping element

1. Self-fixating trial socket for releasable insertion into a corresponding cavity in a hip bone, comprising:
an outer surface (10) matched to the cavity in the hip bone,
an inner surface (11) matched to an inlay and/or to a ball joint head, for receiving the latter in the hip joint socket, and
at least one holding element (13) for fixating the hip joint socket in the cavity, the at least one holding element (13) comprising at least one spring element which is constructed in such a way that in the inserted state the spring element exerts, at least in some places, an outwardly directed spring force on the cavity in the hip bone for releasable fixation of the hip joint socket.

2. Self-fixating trial socket according to claim 1, wherein
the spring element is in the form of a leaf spring (23) which forms the outer surface (10) of the hip joint socket.

3. Self-fixating trial socket according to claim 1, wherein
the proximal end (20) of the holding element (13) is fixed to the hip joint socket in the vicinity of the piercing point of the axis of rotational symmetry thereof, and the position of the distal end (21) located in an edge region (22) of the hip joint socket is variable by being subjected to pressure.

4. Self-fixating trial socket according to claim 1, wherein
the holding element has protrusions (24), on a surface portion of the outer surface, which surface portion faces the hip bone.

5. Self-fixating trial socket according to claim 4, wherein
the protrusions (24) are arranged at the distal end (21) of the holding element (13).

6. Self-fixating trial socket according to claim 1, wherein
in the inserted state of the hip joint socket, a portion thereof projects by at least 0.5 cm beyond the edge of the cavity in the hip bone.

7. Self-fixating trial socket according to claim 1, wherein
the holding element (13) is configured in such a way in the region of the projecting portion that it can be brought into engagement with a corresponding tool for releasing the holding element (13).

8. Self-fixating trial socket according to claim 1, wherein
the hip joint socket has through-apertures such that a grid-like structure is formed.

9. Arrangement comprising the self-fixating trial socket according to claim 1, further comprising a manipulation instrument, the manipulation instrument being forceps-like and constructed in such a way that it is possible for a person or device operating the manipulation instrument to apply to the holding elements (13) a force that is directed into the interior of the hip joint socket.

10. Arrangement comprising the self-fixating trial socket according to claim 1, further comprising a position-determining aid and/or an orientation-determining aid, the position-determining aid and/or orientation-determining aid is fixable to the hip joint socket for determining and/or storing the position and/or orientation thereof.

11. Arrangement comprising the self-fixating test socket according to claim 1 further comprising an inlay which is insertable into the self-fixating test socket.

12. Set consisting of a plurality of self-fixating trial sockets according to claim 1, wherein the individual hip joint sockets differ in size.

13. Set consisting of a plurality self-fixating trial sockets according to claim 12, wherein the individual hip joint sockets differ in size in respect of their ball radius,

14. Arrangement comprising the self-fixating test socket according to claim 11, wherein the inlay is a trial inlay.

15. Self-fixating trial socket according to claim 1, wherein in the inserted state of the hip joint socket, a portion thereof by at least 1.0 cm beyond the edge of the cavity in the hip bone.

16. Self-fixating trial socket according to claim 1, wherein the holding element (13) has sawtooth-like protrusions on a surface portion of the outer surface, which surface portion faces the hip bone.
